(19) United States
(12) Patent Application Publication
(10) Pub. No.: US 2009/0246863 A1
(43) Pub. Date: Oct. 1, 2009

(54) APPARATUS FOR CULTIVATING OIL-RICH MICROALGAE

(76) Inventor: CHIEN-FENG LIN, Hsinchu City (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 12/060,398

(22) Filed: Apr. 1, 2008

(51) Int. Cl. C12M 1/00

(52) U.S. Cl. 435/292.1

(57) ABSTRACT

An apparatus for cultivating oil-rich microalgae includes a tank having a cultivation medium held therein for cultivating the oil-rich microalgae, the tank being provided at predetermined positions with at least one feeding pipe for feeding carbon dioxide and other nutrients to the tank and at least one drain pipe for discharging used cultivation medium from the tank; at least one transparent tubular container partially immersed in the tank for holding a plurality of light-emitting diodes (LEDs) therein; and at least one solar power generating unit being electrically connected to the LEDs in the transparent tubular container. With the above arrangements, the oil-rich microalgae cultivation can be achieved with reduced space and equipment cost without bringing air pollution.

### FIELD OF THE INVENTION

The present invention relates to an apparatus for cultivating oil-rich microalgae, and more particularly to an oil-rich microalgae cultivating apparatus that utilizes solar power generating unit and light-emitting diodes to cultivate oil-rich microalgae.

### BACKGROUND OF THE INVENTION

Due to the worldwide shortage of fossil fuel resource, the price of oil keeps high and will be raised at any time. Moreover, the Kyoto Protocol demands developed nations to limit their greenhouse gas emission. Therefore, it has become a highly important target of the world to positively find alternative energy sources. The alternative energy sources include hydro-power, heat power, wind power, tidal power, geothermal power, solar power, nuclear power, bioenergy, etc. The bioenergy may include bio-diesel, hydrogen, fuel ethanol, etc. Among others, the bio-diesel is one of the most potential alternative energy sources.

Bioenergy is an energy directly derived or transformed from organic substances produced by plants and animals. Since the bioenergy is renewable and useful in reducing waste carbon dioxide, it is highly potential. In most conditions, bioenergy is produced by growing plants, which constantly absorb sunlight to produce organic substances, and the organic substances are then transformed into bioenergy. Therefore, so long as there are plant seeds and sunlight, bioenergy is an energy that would never become depleted.

The plants forming the bioenergy sources absorb carbon dioxide in their growth process, and the carbon dioxide emission from the used bioenergy is lower than the amount of carbon dioxide absorbed by the plants in their photosynthesis. Therefore, the use of bioenergy would not increase the concentration of carbon dioxide in the atmosphere. In fact, the use of bioenergy will decrease the content of carbon dioxide in the air, and is therefore helpful in reducing the greenhouse effect.

Microalgae are one of the most potential sources of bioenergy. In the reports by U.S. Department of Energy, it is mentioned that bio-diesel transformed from the oil produced using the microalgae would fully meet the demand in the American diesel market. Among others, nannochloropsis oculata is one type of oil-rich microalgae. The nannochloropsis oculata is a photoautotroph. It immobilizes carbon dioxide in the process of photosynthesis, and transforms the immobilized carbon dioxide into compounds suitable for mass storage in its body in the form of oil. Therefore, it is possible to extract the rich oil stored in the nannochloropsis oculata for use as a source of producing bio-diesel.

Currently, the microalgae are cultivated in open and widely spread cultivation ponds that require relatively large land areas to obtain sufficient sunlight for the photosynthesis by the microalgae. However, when the cultivation ponds are deep, the sunlight might fail to reach the microalgae grown near the lower portion of the ponds. That is, the microalgae deep in the cultivation ponds could not absorb sufficient sunlight to grow. Moreover, the widely spread cultivation ponds do not allow sufficient agitation of the cultivation medium therein to therefore result in low cultivation efficiency.

It is therefore tried by the inventor to develop an improved apparatus for cultivating oil-rich microalgae within a small area at reduced cost without causing environmental pollution.

### SUMMARY OF THE INVENTION

A primary object of the present invention is to provide an apparatus for cultivating oil-rich microalgae with reduced space and equipment cost without causing air pollution.

To achieve the above and other objects, the apparatus for cultivating oil-rich microalgae according to the present invention includes a tank having a cultivation medium held therein for cultivating the oil-rich microalgae, the tank being provided at predetermined positions with at least one feeding pipe for feeding carbon dioxide and other nutrients to the tank and at least one drain pipe for discharging used cultivation medium from the tank; at least one transparent tubular container for holding a plurality of light-emitting diodes (LEDs) therein; and at least one solar power generating unit being electrically connected to the LEDs in the transparent tubular container.

The transparent tubular container may be a transparent hollow cylinder having an open top. The solar power generating unit is located above the open top of the transparent tubular container, and may be a solar panel. The LEDs may be red LEDs emitting red light having a wavelength between 630 and 675 nanometers or blue-green LEDs emitting blue-green light having a wavelength between 450 and 475 nanometers.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 schematically shows an apparatus for cultivating oil-rich microalgae according to a preferred embodiment of the present invention;

FIG. 2 shows a lighting unit for the oil-rich microalgae cultivating apparatus of the present invention; and

FIG. 3 is a cross sectional view of the lighting unit of FIG. 2.

### DETAILED DESCRIPTION OF THE PREFERRED EMBODIMENTS

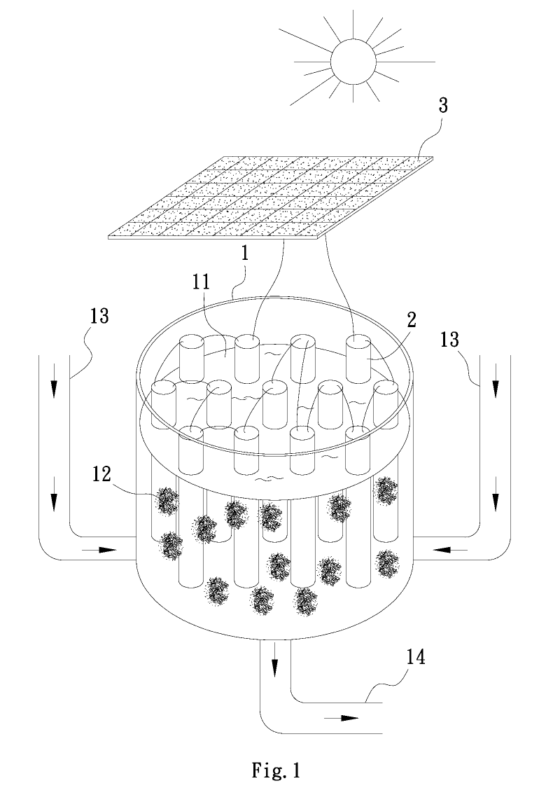

Please refer to FIG. 1 that schematically shows an apparatus for cultivating oil-rich microalgae according to a preferred embodiment of the present invention. As shown, the oil-rich microalgae cultivating apparatus includes a tank 1, at least one transparent tubular container 2, a plurality of light-emitting diodes (LEDs) 4, and at least one solar power generating unit 3.

The tank 1 has a cultivation medium 11 stored therein for cultivating oil-rich microalgae 12. The tank 1 is provided at predetermined positions with one or more feeding pipes 13 and at least one drain pipe 14. Carbon dioxide and other nutrients are fed to the tank 1 via the feeding pipes 13, and used cultivation medium 11 is discharged via the drain pipe 14. The transparent tubular container 2 may be a transparent hollow cylinder, in which the LEDs 4 are received. The solar power generating unit 3 may be a solar panel being connected to the LEDs 4 in series or in parallel.

The transparent tubular container 2 is partially immersed in the cultivation medium 11. The LEDs 4 emit red light or blue-green light. The red light has a wavelength between 630 and 675 nanometers, and the blue-green light has a wavelength between 450 and 475 nanometers. With these wavelengths, the oil-rich microalgae 12 may photosynthesize more efficiently.

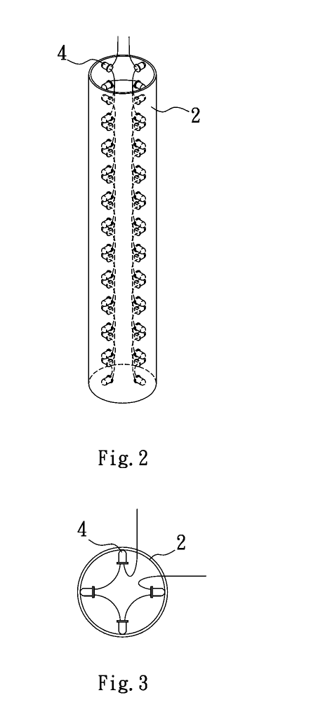

A plurality of transparent tubular containers 2 and the LEDs 4 received therein together form lighting units for the oil-rich microalgae cultivating apparatus of the present invention. FIG. 2 shows one lighting unit for the present invention. As shown, one transparent tubular container 2 may have a plurality of LEDs 4 received therein. The LEDs 4 in each transparent tubular container 2 may be arranged in multiple spaced layers with the LEDs 4 in each layer being equally spaced to direct in different directions. The solar power generating unit 3 is located above the lighting units and electrically connected to the LEDs 4 while the transparent tubular containers 2 are partially immersed in the cultivation medium 11. The solar power generating unit 3 absorbs sunlight and supplies electric power to the LEDs 4 for the latter to emit light, allowing the oil-rich microalgae 12 in upper and lower portions of the tank 1 to photosynthesize at the same time.

Since the transparent tubular containers 2 are partially immersed in the cultivation medium 11 in an upright position and the LEDs 4 are received in the tubular containers 2 in multiple spaced layers and directed in different directions, even the oil-rich microalgae 12 grown at the lower portion of the tank 1 can absorb light from the LEDs 4 and are allowed to photosynthesize. That is, with the present invention, only a small area is required to cultivate more oil-rich microalgae 12.

The use of the solar power generating unit 3 to supply electric power to the LEDs 4 enables lowered production cost and minimized environmental pollution.

Further, in the present invention, carbon dioxide is fed to the tank 1 as nutrient for the microalgae 12 to grow. By doing this, the content of carbon dioxide in the ambient air is also decreased to effectively reduce the air pollution.

The LEDs 4 have extended usable life and enhanced brightness to increase the growth of the oil-rich microalgae 12.

With the above arrangements, the apparatus for cultivating oil-rich microalgae according to the present invention is novel, improved, and industrially valuable for use. In the present invention, the use of the tank to hold the cultivation medium and the transparent tubular containers, and the use of the solar power generating unit to supply electric power to the LEDs for the same to emit highly bright light over a long time together enable the microalgae cultivation to be achieved with reduced space and equipment cost without bringing air pollution. Therefore, products derived from the present invention would no doubt fulfill the current market demand.

What is claimed is:

1. An apparatus for cultivating oil-rich microalgae, comprising:
a tank having a cultivation medium held therein for cultivating the oil-rich microalgae, the tank being provided at predetermined positions with at least one feeding pipe for feeding carbon dioxide and other nutrients to the tank and at least one drain pipe for discharging used cultivation medium from the tank;
at least one transparent tubular container partially and vertically immersed in the tank for holding a plurality of light-emitting diodes (LEDs) therein; and
at least one solar power generating unit being electrically connected to the LEDs in the transparent tubular container.

2. The apparatus for cultivating oil-rich microalgae as claimed in claim 1, wherein the transparent tubular container is a transparent hollow cylinder.

3. The apparatus for cultivating oil-rich microalgae as claimed in claim 1, wherein the solar power generating unit is a solar panel.

4. The apparatus for cultivating oil-rich microalgae as claimed in claim 2, wherein the solar power generating unit is a solar panel.

5. The apparatus for cultivating oil-rich microalgae as claimed in claim 1, wherein the LEDs are selected from the group consisting of red LEDs and blue-green LEDs.

6. The apparatus for cultivating oil-rich microalgae as claimed in claim 5, wherein the red LEDs emit red light having a wavelength between 630 and 675 nanometers, and the blue-green LEDs emit blue-green light having a wavelength between 450 and 475 nanometers.

7. The apparatus for cultivating oil-rich microalgae as claimed in claim 4, wherein the LEDs are selected from the group consisting of red LEDs and blue-green LEDs.

8. The apparatus for cultivating oil-rich microalgae as claimed in claim 7, wherein the red LEDs emit red light having a wavelength between 630 and 675 nanometers, and the blue-green LEDs emit blue-green light having a wavelength between 450 and 475 nanometers.
